(19) United States
(12) Patent Application Publication
(10) Pub. No.: US 2006/0167538 A1
(43) Pub. Date: Jul. 27, 2006

(54) Inflatable biliary stent

(76) Inventor: Brian K. Rucker, King, NC (US)

Correspondence Address: BRINKS HOFER GILSON & LIONE/CHICAGO/COOK, PO BOX 10395, CHICAGO, IL, 60610, US

(21) Appl. No.: 11/316,283

(22) Filed: Dec. 21, 2005

Related U.S. Application Data
(60) Provisional application No. 60/638,889, filed on Dec. 23, 2004.

(51) Int. Cl. A61F 2/06

(52) U.S. Cl. 623/1.25

(57) ABSTRACT

An inflatable stent is provided for placement within the biliary duct to facilitate drainage therethrough and maintain the patency of the duct. The biliary stent includes a tubular member, an inflatable reservoir and a port located at a proximal portion of the tubular member. The tubular member has interior and exterior surfaces and an inflatable reservoir disposed circumferentially between the interior and exterior surfaces. An inflation fluid, such as a liquid or gas, may be inserted through the port and contained within the inflatable reservoir to expand the diameter of the biliary stent. The biliary stent may be removed from the patient by deflating the inflatable reservoir and using a removal device such as a forceps or snare.

### PRIORITY CLAIM

This application claims priority to U.S. Provisional Patent Application Ser. No. 60/638,889, entitled “Inflatable Biliary Stent,” filed Dec. 23, 2004. The disclosure of the above application is incorporated herein by reference in its entirety.

### BACKGROUND

1. Technical Field

The present invention is directed to stents that are implantable in a vessel or duct within the body of a patient to maintain patency, and in particular, to an inflatable stent that may be used in biliary ducts.

2. Background Information

Stents may be inserted into an anatomical vessel or duct to maintain or restore patency in a formerly blocked or constricted passageway. Stents may be manufactured using materials such as plastic or metal, and may comprise a variety of configurations, for example, a wire-mesh, coil or helical shape, or a slotted tube configuration.

There are different methods for implanting stents in the human body. Some stents may be delivered to a target site in a compressed configuration and subsequently expanded by removing a compression sheath. In such embodiments, a shape-memory alloy such as nitinol may be employed to cause the stent to return to a predetermined configuration upon removal of the sheath.

Other stents may be balloon-expandable. A delivery system may include, for example, a catheter having proximal and distal ends and a balloon disposed on the distal end of the catheter. The stent may be coupled to the balloon during insertion towards a target location. The delivery system comprises a smaller delivery profile than the diameter of the vessel into which the stent is implanted. The catheter may be inserted over a wire guide into a vessel or duct and advanced until the stent is aligned at the target site. The stent then may be deployed by inflating the balloon to expand the stent diameter, whereby the stent engages and may slightly expand the lumen diameter of the vessel or duct.

A stent should have adequate strength in the deployed state to sustain the natural tendency of the vessel wall to recoil. If the stent recoils after being deployed, it may become dislodged and travel to an undesired location in the vessel or duct.

Some implanted stents may be removed and replaced over time. Removal of an implanted stent may present discomfort to the patient, and may cause internal bleeding or scarring of the vessel or duct. Depending on the material construction of the stent, e.g., whether it is plastic or a metal mesh, an implanted stent may be relatively difficult to remove.

Therefore, there is a need for an improved stent that may be inserted or deposited in the biliary duct and may be expanded to a diameter that maintains patency of the duct. In addition, there is a further need for an inflatable stent that does not have to be removed frequently, or in the event it needs to be removed, it may be removed with relative ease.

### SUMMARY

The present invention is directed to an inflatable biliary stent that facilitates drainage through the biliary duct and maintains patency of the duct. The invention utilizes an inflatable reservoir that may be inflated to provide an expanded stent configuration. The inflatable reservoir may be subsequently deflated to facilitate withdrawal of the stent from the duct. Although applications relating to biliary stenting are discussed in detail herein, the stent and methods of the present invention may be used for any passageway of the patient's body including, but not limited to, arteries, veins, urethras, and so forth.

In one embodiment of the present invention, an inflatable biliary stent comprises an elongate tubular member having proximal and distal ends, interior and exterior surfaces, and an inner lumen disposed within the interior surface. The tubular member may comprise different configurations, for example, in the form of a hollow tubular stent or a spiral-shaped stent.

An inflatable reservoir is disposed circumferentially between the interior and exterior surfaces of the tubular member. The stent has a first non-inflated configuration that facilitates insertion into a biliary duct and a second configuration that presses radially outward against the ductile wall to maintain the stent in position. In a preferred embodiment, a port adapted for inserting an inflation fluid is located at the proximal end of the elongate tubular member. The port is configured to retain injected fluid within the reservoir and may comprise, for example, a self-sealing membrane or a one-way valve.

In the context of the present invention, the term “inflation fluid” may encompass any liquid, gas, resin material or other deliverable substance. In one embodiment, a liquid or gas may be injected through the port to fill the inflatable reservoir, thereby expanding the reservoir and changing the stent from a delivery configuration to an expanded configuration. In operation, the stent may be inserted into the biliary duct using a wire guide and a balloon catheter. In a first step, an endoscope may be directed into a patient's duodenum, then the wire guide is inserted through a working lumen of the endoscope and into the biliary duct. The balloon catheter, with the stent coupled thereto, may be inserted over the wire guide. When positioned at a desired location, the balloon may be expanded to expand the stent. The balloon may be held in the expanded state while fluid is injected into the inflatable reservoir via the port of the stent. At a later time, the inflatable reservoir may be deflated to facilitate withdrawal of the stent from the duct.

In an alternative embodiment, the inflation fluid may comprise a curable resin material. The curable resin may be loaded into the inflatable reservoir prior to insertion of the stent into the patient's biliary duct. When the balloon catheter is positioned at the target location, the balloon is deployed to expand the stent against the lumen of the biliary duct. While the balloon holds the stent against the ductile wall, a light may be delivered to the curable resin within the stent, e.g., via the balloon, to cure the material into a final polymerized state. In effect, the stent is held in its desired shape against the ductile wall.

Other systems, methods, features and advantages of the invention will be, or will become, apparent to one with skill in the art upon examination of the following figures and detailed description. It is intended that all such additional systems, methods, features and advantages be within the scope of the invention, and be encompassed by the following claims.

### BRIEF DESCRIPTION OF THE DRAWINGS

The invention can be better understood with reference to the following drawings and description. The components in the figures are not necessarily to scale, emphasis instead being placed upon illustrating the principles of the invention. Moreover, in the figures, like referenced numerals designate corresponding parts throughout the different views.

FIG. 1 is a side view of a first embodiment of a biliary stent provided in accordance with principles of the present invention;

FIG. 2 is a side-sectional view of the biliary stent shown in FIG. 1;

FIG. 3 is a cross-sectional view of the biliary stent of FIG. 1 taken along line 3-3;

FIG. 4 is a side view of a second embodiment of a biliary stent provided in accordance with principles of the present invention;

FIG. 5 is a side-sectional view of the biliary stent shown in FIG. 4;

FIG. 6 is a cross-sectional view of the biliary stent of FIG. 4 taken along line 6-6; and

FIGS. 7A-7E schematically illustrate method steps for deploying the biliary stent of FIGS. 1-3 at a desired location in a biliary duct.

### DETAILED DESCRIPTION OF THE PREFERRED EMBODIMENTS

Turning now to the figures, reference numbers are used to designate corresponding elements in the figures. Although the present invention will be described with reference to preferred embodiments, those skilled in the art will recognize that changes may be made in form and detail without departing from the spirit and scope of the invention. As such, it is intended that the following detailed description be regarded as illustrative rather than limiting and that it is the appended claims, including all equivalents thereof, which are intended to define the scope of the invention

In the present application, the term “proximal” refers to a direction that is generally towards a physician during a medical procedure, while the term “distal” refers to a direction that is generally towards a target site within a patient's anatomy during a medical procedure.

Further, although the invention describes use of a stent that may be deployed in a biliary duct to treat a biliary occlusion, the inflatable stent of the present invention may be used with any passageway of the human body including, but not limited to, arteries, veins, urethras, and so forth.

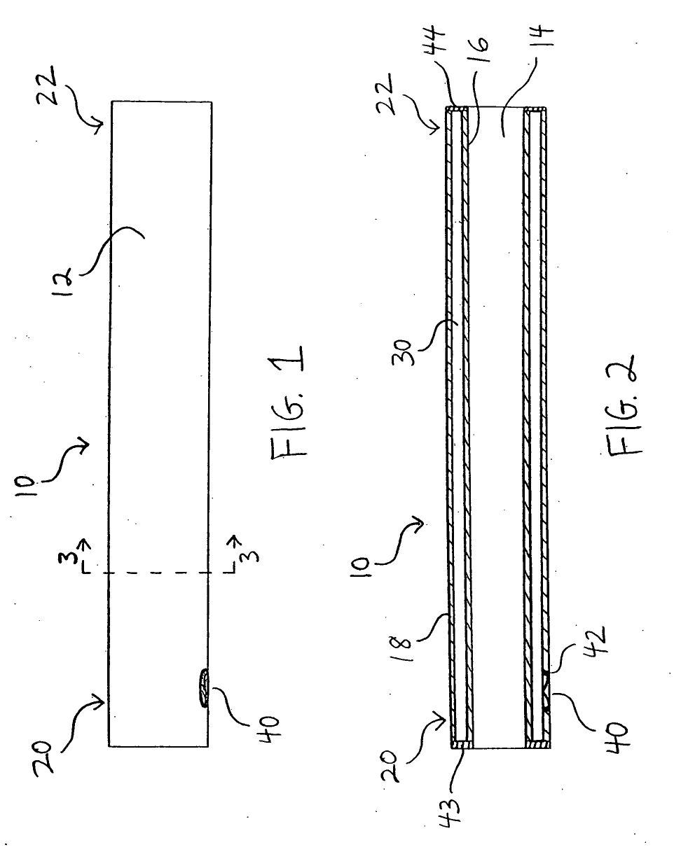

Referring now to FIGS. 1-3, a first embodiment of the present invention is described. In FIGS. 1-3, inflatable biliary stent 10 comprises elongate tubular member 12, inflatable reservoir 30 and at least one port 40. Elongate member 12 is generally cylindrical in shape and comprises proximal end 20 and distal end 22. Elongate member 12 further comprises interior surface 16, exterior surface 18, and inner lumen 14 extending longitudinally within interior surface 16, as shown in FIG. 2.

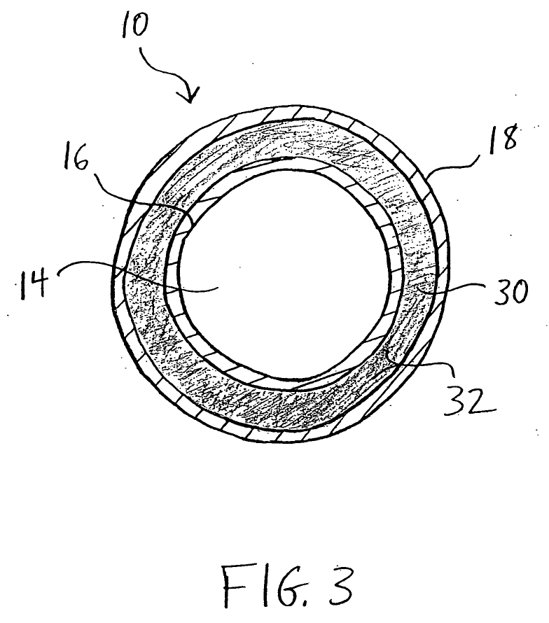

Inflatable reservoir 30 is circumferentially disposed between interior surface 16 and exterior surface 18, as shown in FIGS. 2-3. Elongate member 12 may taper at its proximal or distal end, if desired, or may maintain a continuous diameter along its entire length, as generally depicted.

The interior and exterior surfaces of elongate member 12 may be formed of two substantially concentric, tubular layers of material. Alternatively, interior and exterior surfaces 16 and 18 may be formed from one tubular material, wherein inflatable reservoir 30 is annularly formed therein. Proximal and distal end caps 43 and 44 may be employed to seal the end regions of elongate tubular member 12, as shown in FIG. 2, thereby preventing fluid from escaping reservoir 30. Alternatively, the proximal ends of interior surface 16 and exterior surface 18 may be fused directly together to prevent proximal leakage, while the distal ends of interior surface 16 and exterior surface 18 also may be fused together to prevent distal leakage. The end regions may be reinforced to ensure that fluid will not leak.

Interior and exterior surfaces 16 and 18 may be formed from various suitable materials. For example, the materials may comprise one or more layers of expandable material such as polyurethane, radiopaque polyurethane material, polyethylene terephthalate, silicone, natural rubber, synthetic rubber, nylon, latex, polyethylene, an elastic material, or combinations thereof.

The material compositions of interior and exterior surfaces 16 and 18 preferably are such that stent 10 will retain substantially the same configuration depicted in FIG. 2 during inflation of reservoir 30. In particular, interior surface 16 will not bulge substantially inward during inflation of reservoir 30, and exterior surface 18 will not bulge substantially outward. Therefore, the configuration of stent 10 in the delivery state is substantially similar to the configuration in the deployed state, with the exception that the expanded diameter is substantially proportionally larger than the delivery diameter. Importantly, since interior surface 16 will not bulge substantially inward, inner lumen 14 will not become substantially occluded during or after inflation of stent 10.

Stent 10 further comprises port 40, which may be disposed in a portion of exterior surface 18 near proximal end 20 of elongate member 12. Port 40 is configured for fluid communication with reservoir 30 and is sufficiently sized to permit the insertion or withdrawal of inflation fluid from reservoir 30. Port 40 may be flush or protrude from the exterior surface of elongate member 12. In one embodiment, port 40 is comprised of a self-sealing membrane that may be accessed using a catheter needle or other means for injecting fluid through the membrane. Such a self-sealing membrane may comprise an oval or circular shape that tapers inward to prevent leakage of inflation fluid, when the fluid is disposed within reservoir 30. Port 40 also may be sealed using an external component that is semi-permeable or porous to permit the insertion or withdrawal of the inflation fluid using a catheter needle. Adhesive material 42 may circumferentially surround port 40 to reduce or prevent leakage of the inflation fluid.

Alternatively, port 40 may comprise a device that extends at least partially outward from exterior surface 18 and regulates the ingress and egress of inflation fluid. For example, a one-way valve may be used to regulate insertion of fluid directly into reservoir 30, while a secondary device is required to extract fluid from reservoir 30 for purposes of deflating stent 10.

As will be described in greater detail below, when inflation fluid 32 (see FIG. 3) is inserted into reservoir 30 through port 40, elongate tubular member 12 may expand in a radially outward direction. Therefore, in areas where a biliary duct is occluded, the expanded stent may help maintain the patency of the duct by permitting flow through inner lumen 14. The amount of inflation fluid 32 inserted into reservoir 30 may vary as needed to permit stent 10 to securely fit within a patient's biliary duct.

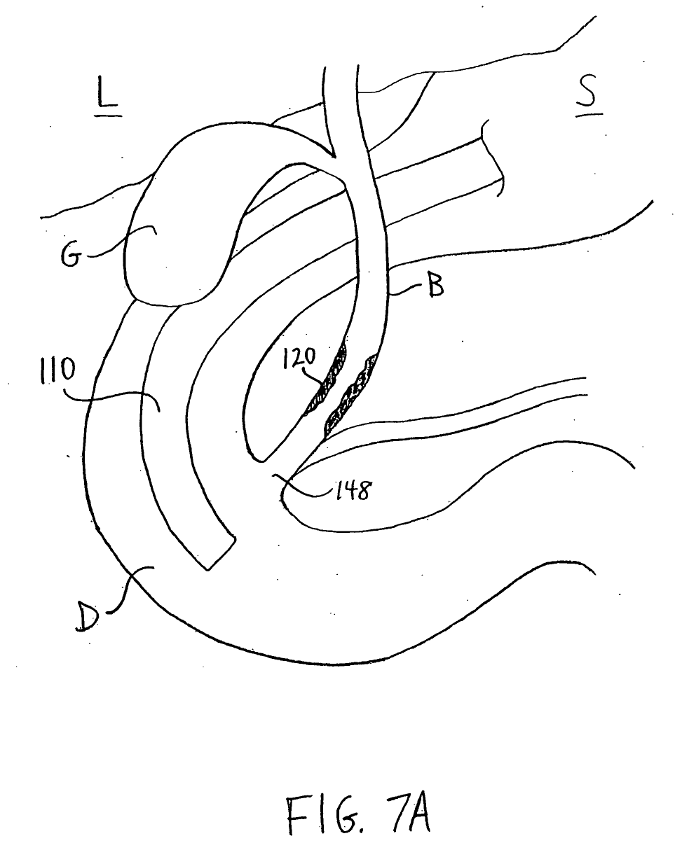

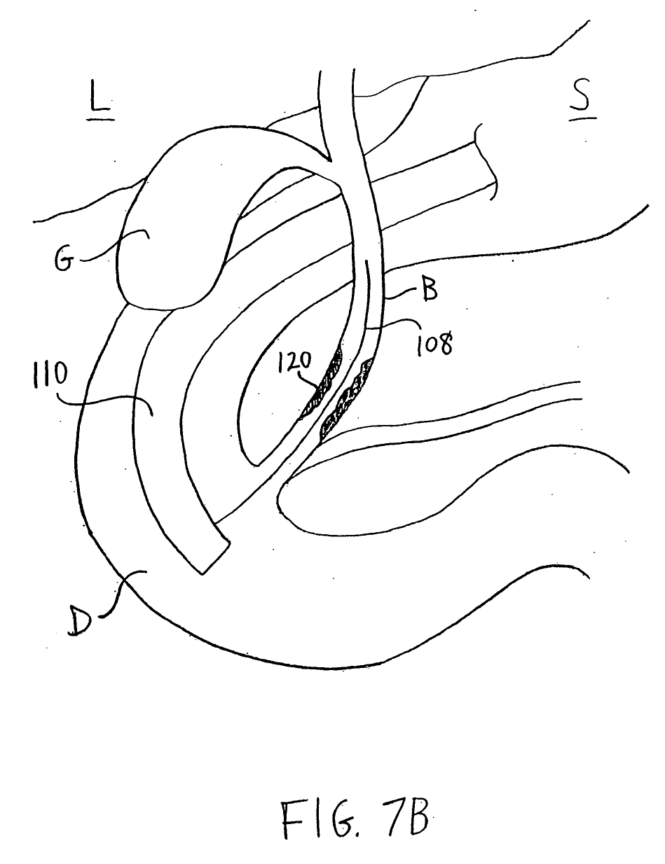

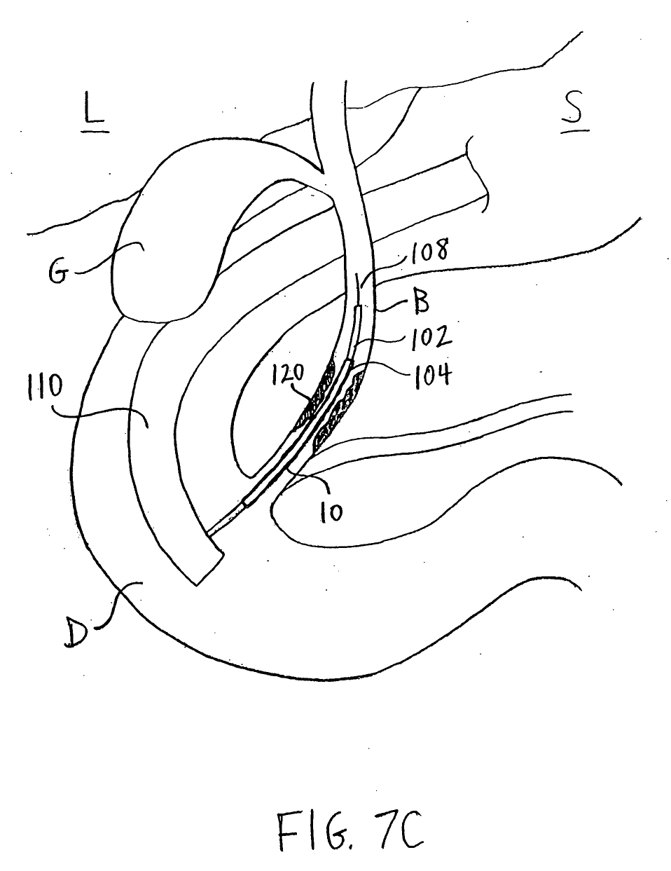

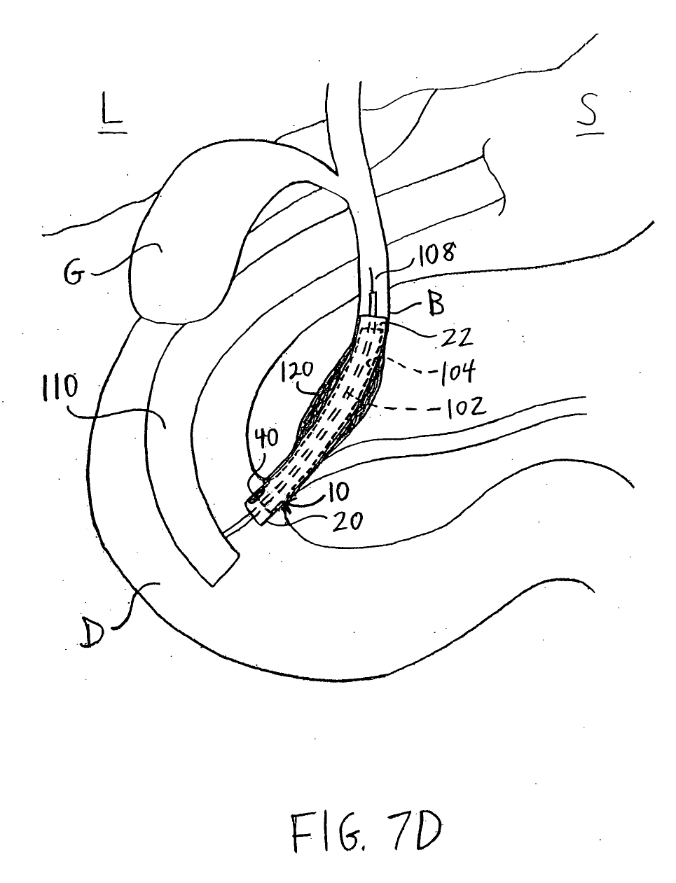

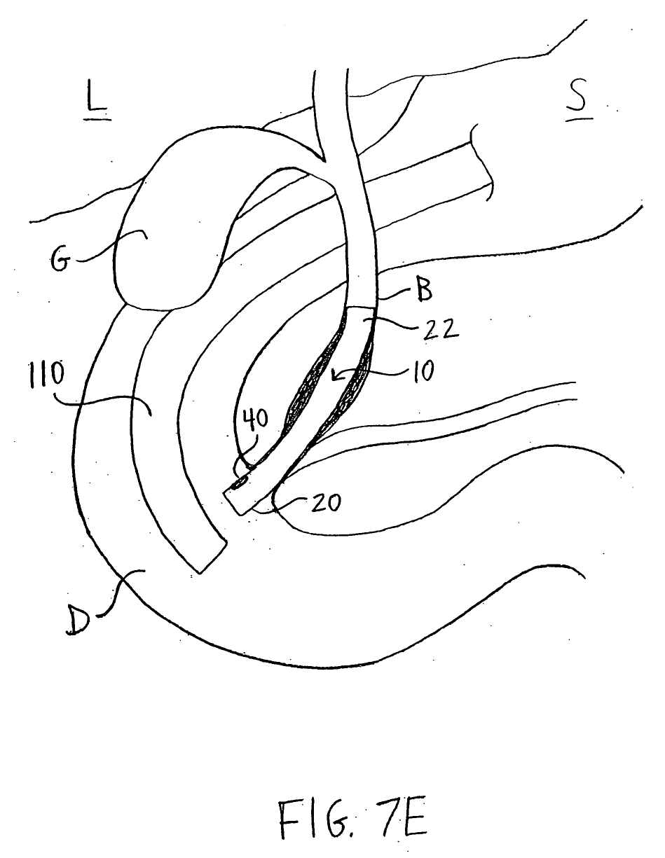

As will be described in greater detail below with respect to FIGS. 7A-7E, reservoir 30 remains in a deflated delivery configuration so that stent 10 may be delivered to a target location. In one embodiment described below, stent 10 may be delivered into the biliary duct by advancing a balloon catheter over a wire guide, wherein the wire guide and catheter are advanced through a working lumen of an endoscope disposed in the duodenum. The stent may be coupled to the balloon in its reduced profile, then advanced through the endoscope and into the biliary duct.

After stent 10 is delivered to a target location, it is expanded to a desired configuration, as described with respect to FIGS. 7D-7E below. In the expanded configuration, stent 10 presses radially outward against the ductile wall to maintain patency of the duct. In the deflated configuration, an overall outer diameter of stent 10 may be about 1-4 mm, but after inflation of reservoir 30, the overall outer diameter of stent 10 may expand to about 9-11 mm.

As noted above, the term “inflation fluid” may encompass any liquid, gas, resin material or other deliverable substance. In one embodiment, inflation fluid 32 may comprise liquids or gases, such as water, saline, air, or contrast media that affords radiographic visualization.

In an alternative embodiment, inflation fluid 32 may comprise a pliable, light-curable resin. Preferably, the resin material is responsive to a selective wavelength to initiate curing. In this configuration, resin materials may be prepared externally and then injected into reservoir 30 via port 40 prior to insertion of stent 10. The stent may be delivered using a balloon catheter, wherein the balloon is constructed of a material that may transmit ultraviolet light to permit exposure of the resin to the light. Therefore, the curing process may be performed in vivo, while the balloon holds the stent in a desired configuration within the biliary duct, as shown with respect to FIG. 7D below.

Resins can be cured by mixing a monomer, e.g., an acrylic, with an initiator, e.g., a peroxide, the curing rate of which is controlled by the proportion of materials. The resulting thermoplastic has a glass transition temperature above which the material is flexible, and below which the material may be rigid, glassy and/or brittle. Therefore, a thermoplastic with a glass transition temperature above body temperature, but below a tissue damaging temperature, could be heated in vivo to facilitate removal of stent 10.

Alternatively, a thermosetting polymer can be cured as a one-component system using moisture or heat or oxygen instead of a two-component system which involves combing two resins that react with each other, e.g., cross-link, to form a three-dimensional solid. Common thermosets include amines, urethanes, polyesters, epoxies, and polyimides. However, thermosets can not be softened, thus removal of a thermoset stent requires breaking it into pieces and removing each piece from the biliary duct.

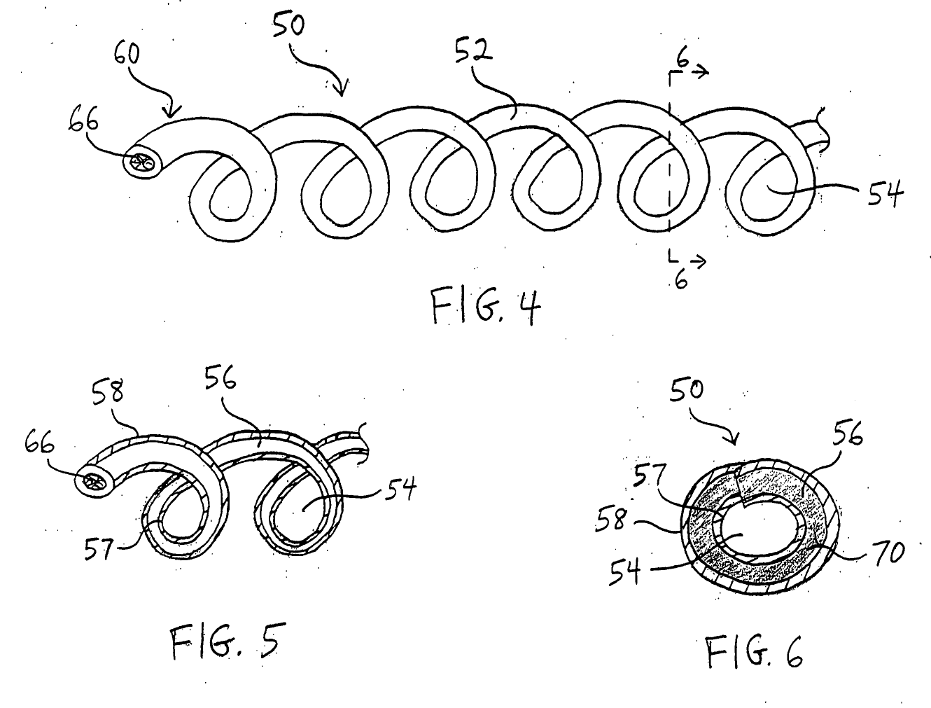

Referring now to FIGS. 4-6, an alternative configuration of an inflatable biliary stent provided in accordance with principles of the present invention is described. In FIG. 4, stent 50 comprises spiral-shaped tubular member 52 having reservoir 56 and port 66. While a spiral-shaped tubular member is depicted, stent 50 may have other configurations.

In the embodiment of FIGS. 4-6, reservoir 56 is disposed between interior surface 57 and exterior surface 58 of stent 50, as shown in FIGS. 5-6. In this embodiment, tubular member 52 may comprise, for example, one or more layers of expandable material such as polyurethane, radiopaque polyurethane material, polyethylene terephthalate, silicone, natural rubber, synthetic rubber, nylon, latex, polyethylene, an elastomeric material, or combinations thereof.

Reservoir 56 and port 66 perform substantially similar functions as reservoir 30 and port 40, respectively, as described above with respect to FIGS. 1-3. For example, port 66 may comprise a self-sealing membrane or one-way valve that may be used in conjunction with a secondary device, such as a catheter needle, to infuse fluid through port 66 for retention within reservoir 56. In the embodiment of FIGS. 4-6, port 66 is illustratively disposed within an end surface at proximal end 60 of stent 50. Alternatively, the port may be disposed within a lateral surface of proximal end 60, e.g., in the manner that port 40 is disposed within exterior surface 18 of stent 10.

Stent 50 may be inflated using a liquid or gas, or alternatively, by balloon expanding the stent and curing a resin disposed within reservoir 56, as described hereinabove with respect to stent 10 of FIGS. 1-3. In the expanded configuration, the spiral-shaped design of stent 50 is configured to maintain patency in a biliary duct by allowing flow through inner passageway 54, as shown in FIGS. 4-6. Like the embodiment of stent 10 above, stent 50 also may be removed from the biliary duct upon deflation by removing inflation fluid 70 from reservoir 56.

Referring now to FIGS. 7A-7E, method steps for deploying biliary stent 10 of FIGS. 1-3 at a desired location in a biliary duct are described. As shown in FIG. 7A, occlusion 120 has formed within biliary duct B. In a first step, endoscope 110 may be inserted into a patient's mouth, through the esophagus, through stomach S, and into duodenum D, as schematically shown in FIG. 7A. In the event a biliary occlusion exists, a sphincterectomy may be performed at sphincter of Oddi 148 using endoscope 110. The sphincterectomy may facilitate access into biliary duct B to perform further functions described below.

Referring now to FIG. 7B, wire guide 108 is inserted through a working lumen (not shown) of endoscope 110. For example, a working lumen of endoscope 110 may have a diameter of about 3-4 mm, while the overall diameter of endoscope 110 may be about 10-14 mm. In operation, wire guide 108 is advanced distally through the working lumen into biliary duct B. The wire guide is carefully advanced through occlusion 120 and disposed distal to the occlusion, as shown in FIG. 7B.

Referring now to FIG. 7C, in a next step catheter 102 having proximal and distal ends and balloon 104 disposed on the distal end is inserted over wire guide 108 into biliary duct B. Catheter 102 is advanced distally through the working lumen of the endoscope, into biliary duct B, and the distal end is disposed just distal to occlusion 120, as shown in FIG. 7C. In a preferred embodiment, stent 10 is securely coupled to balloon 104 as the catheter is advanced into the biliary duct. Alternatively, the stent may be subsequently inserted into the biliary duct using a second instrument (not shown), for example, after balloon dilation has been performed on occlusion 120.

Referring to FIG. 7D, after catheter 102 having balloon 104 and stent 10 coupled thereto have been placed at a desired location, balloon 104 is inflated to cause localized radial expansion of biliary duct B. Catheter 102 may comprise an inflation lumen (not shown) that is in fluid communication with balloon 104, thereby permitting the physician to selectively inflate and deflate the balloon.

When balloon 104 is partially or fully inflated, stent 10 may be temporarily held in place. In one embodiment, described above, inflation fluid 32 comprises a curable resin that is pre-loaded into reservoir 30 prior to insertion of stent 10 into biliary duct B. In this embodiment, the material may be cured into a polymer in vivo, i.e., while balloon 104 holds stent 10 in a desired expanded configuration. As described above, the curable resin may be cured by the provision of a suitable light, which may be delivered through balloon 104. Alternatively, other curing techniques may be employed to cause the material to harden in the expanded state. Once the injected material is cured and the stent is retained in the expanded diameter, balloon 104 is deflated and catheter 102 and wire guide 108 are subsequently removed from the patient's body via endoscope 110. As shown in FIG. 7E, stent 10 is retained within biliary duct B and provides a radially outward force sufficient to maintain patency within the duct. In a final step, endoscope 110 is removed from the patient's body.

In an alternative method, inflation fluid may be injected through port 40 into reservoir 30 in vivo, thereby causing stent 10 to enlarge in diameter, as described above. For example, while stent 10 is temporarily held in place by expanded balloon 104, as shown in FIG. 7D, liquid or gases may be delivered through port 40 via an injection means (not shown), such as a catheter needle, thereby inflating reservoir 30 in vivo. Either during or after the process of inflating reservoir 30 via port 40, balloon 104 may be deflated. The inflated stent will be held against an inner lumen of biliary duct B, as shown in FIG. 7E, due the expansion of reservoir 30. The inflation means, along with catheter 102 having balloon 104 deflated, then are removed from the patient via endoscope 110.

If it becomes desirable to remove inflated stent 10 from the patient, another endoscope 110 subsequently may be inserted into duodenum D. A suction needle (not shown) may be advanced distally through a working channel of endoscope 110, and the needle may access port 40 to remove inflation fluid from reservoir 30. In the event that a curing material is cured within reservoir 30, the material may be heated in vivo to facilitate removal of stent 10, as described above. After the stent has been sufficiently deflated, a forceps or snare may be used to extract stent 10 from the patient's body via the endoscope.

While various embodiments of the invention have been described, it will be apparent to those of ordinary skill in the art that many more embodiments and implementations are possible within the scope of the invention. Accordingly, the invention is not to be restricted except in light of the attached claims and their equivalents.

I claim:

1. A stent comprising:
an elongate tubular member having proximal and distal ends, interior and exterior surfaces, and an inner lumen disposed within the interior surface;
an inflatable reservoir circumferentially disposed between the interior and exterior surfaces of the elongate tubular member; and
a port disposed in a portion of the exterior surface, the port configured for selective fluid communication with the inflatable reservoir,
wherein an inflation fluid may be inserted into the inflatable reservoir, via the port, to cause the elongate tubular member to transform from a reduced delivery configuration to an expanded deployed configuration.

2. The stent of claim 1 wherein the stent is insertable into a biliary duct.

3. The stent of claim 1 wherein the port comprises a one-way valve configured to permit insertion of the inflation fluid into the inflatable reservoir.

4. The stent of claim 1 wherein the port comprises a self-sealing membrane.

5. The stent of claim 1 wherein the stent is configured for delivery into a biliary duct using a catheter having proximal and distal ends and a balloon disposed on the distal end of the catheter.

6. The stent of claim 1 wherein the inflation fluid comprises a liquid.

7. The stent of claim 1 wherein the inflation fluid comprises a curable resin material.

8. A method of deploying a stent, the method comprising:
providing a stent comprising a tubular member having proximal and distal ends, interior and exterior surfaces, and an inner lumen disposed within the interior surface;
employing a delivery system to deliver the stent to a target location; and
inserting an inflation fluid into an inflatable reservoir, the inflatable reservoir disposed circumferentially between the interior and exterior surfaces of the tubular member,
wherein insertion of the inflation fluid into the inflatable reservoir causes the stent to transform from a reduced delivery configuration to an expanded deployed configuration.

9. The method of claim 8 wherein the stent is deployed in a biliary duct.

10. The method of claim 9 wherein the expanded deployed configuration is suitable for maintaining patency in a portion of the biliary duct.

11. The method of claim 8 wherein inserting an inflation fluid further comprises delivering the inflation fluid through a port disposed in a portion of the exterior surface, the port configured to selectively permit fluid communication with the inflatable reservoir.

12. The method of claim 8 wherein inserting an inflation fluid further comprises inserting a liquid.

13. The method of claim 8 wherein the port comprises a one-way valve, the method further comprising permitting the injection of the inflation fluid into the inflatable reservoir via the one-way valve.

14. The method of claim 8 wherein the delivery system comprises a wire guide, a catheter having proximal and distal ends, and a balloon mounted on the distal end of the catheter, the method further comprising:
inserting the wire guide into a biliary duct;
advancing the catheter over the wire guide to a target location in the biliary duct; and
inflating the balloon of the catheter to cause the stent to radially expand.

15. The method of claim 14 further comprising maintaining the balloon in an inflated state while fluid is delivered through the port.

16. The method of claim 14 further comprising curing the inflation fluid while the balloon maintains the stent in a radially expanded configuration.

17. The method of claim 8 further comprising subsequently removing the stent by deflating the inflatable reservoir.

18. A stent comprising:
a spiral-shaped tubular member having proximal and distal ends, interior and exterior surfaces, and an inner passageway disposed within the interior surface;
an inflatable reservoir circumferentially disposed between the interior and exterior surfaces of the spiral-shaped tubular member; and
a port disposed in a portion of the exterior surface, the port configured for fluid communication with the inflatable reservoir,
wherein an inflation fluid may be inserted into the inflatable reservoir, via the port, to cause the spiral-shaped tubular member to transform from a reduced delivery configuration to an expanded deployed configuration.

19. The stent of claim 18 wherein the stent is insertable into a biliary duct.

20. The stent of claim 18 wherein the port comprises a one-way valve, the one-way valve permitting the insertion of the inflation fluid into the inflatable reservoir.

21. The stent of claim 18 wherein the port comprises a self-sealing membrane.

22. The stent of claim 18 wherein the stent is configured for delivery into a biliary duct using a catheter having proximal and distal ends and a balloon disposed on the distal end of the catheter.

23. The stent of claim 18 wherein the inflation fluid comprises a liquid.

24. The stent of claim 18 wherein the inflation fluid comprises curable resin material.
